(19) United States
(12) Patent Application Publication
(10) Pub. No.: US 2014/0221765 A1
(43) Pub. Date: Aug. 7, 2014

(54) PATIENT/PROFESSIONAL-COLLABORATIVE, TRADITIONAL-DIAGNOSIS-BYPASSING, MENTAL HEALTH THERAPY SYSTEM AND METHODOLOGY

(71) Applicant: Integrated Health Pathways, LLC, Portland, OR (US)

(72) Inventors: David Lynn Harmon, Portland Park, OR (US); Barbara Wexler, Portland, OR (US); Alois Frank Fee, Fulshear, TX (US); Tzu-Chieh Yang, Portland, OR (US); Peter Galen, Portland, OR (US)

(73) Assignee: Integrated Health Pathways, LLC, Portland, OR (US)

(21) Appl. No.: 14/173,706

(22) Filed: Feb. 5, 2014

Related U.S. Application Data
(60) Provisional application No. 61/762,228, filed on Feb. 7, 2013.

(51) Int. Cl. A61B 5/16; A61B 5/00

(52) U.S. Cl. CPC A61B 5/165; A61B 5/742
USPC 600/300

(57) ABSTRACT

An traditional-diagnosis-bypassing method for improving a mental health patient-therapy program through incorporating in it, centrally, a patient's expressed therapy goals, including (a) accessing an electronic information system which is programmed with information-capturing and related-question-based, guiding software associated with patient-personalizing therapy planning, structuring and implementing, (b) encouraging a collaboration dialogue between patient and professional to capture information relating to the patient's mental health symptoms and associated, patient-expressed therapy goals, and collaboratively entering related dialogue information into the system, and (c) based upon such information entering, and through professional-and-patient interactive engagement with the system, and functioning expressly in a conventional-diagnosis-bypassing, question and answer mode which is bottomed centrally upon recognizing the patient's expressed therapy goals, collaboratively designing, building, and thereafter implementing, a patient-specific therapy.

### CROSS-REFERENCE TO RELATED APPLICATION

This application claims filing-date priority to currently copending U.S. Provisional Patent Application Ser. No. 61/762,228, filed Feb. 7, 2013, for “Computer-and-Web-Based, Mental Health Therapy Guidance”, the entire disclosure content in which prior application is hereby incorporated herein by reference.

### BACKGROUND AND SUMMARY OF THE INVENTION

This invention pertains to the field of mental health, behavioral-transformation therapy, and in particular, to a software-supported, traditional-diagnosis-bypassing methodology and system aimed at collaborating a mental health professional and a patient in the building, implementing, modifying, and history-and-progress tracking of an effective, patient-tailored, and thereby improved-accuracy, mental health treatment plan. The terms “therapy” and “treatment” are used interchangeably and synonymously herein. The term “patient-tailored” recognizes the important invention feature involving emphasizing therapy-plan designing and implementing with a key focus directed to what a patient expressly wishes to accomplish. This “patient-tailored” feature offers a therapy approach which is strikingly different from the more conventionally created, “top-down”, traditional, professional-diagnostic approach. With this significant, “patient-tailored” focus in place, the patient becomes a de facto central figure in the creation and execution of an effective, personally designed and implemented therapy.

As will become apparent with respect to the “patient-tailored” concept just mentioned, the system, its associated software, and the linked methodology, of the present invention exude a therapy-practice patient-personalizing nature which encourages professional adherence to centralization of a patient's therapy-result desires.

Software programming provided in the system of the present invention for implementing methodologic practice of the invention, may be constructed in a variety of different ways, all well within the skill levels of those generally skilled in the art of programming, and accordingly, specific details of this programming are not set forth herein since they form no part of the present invention.

In the background landscape respecting the present invention, the field of mental health therapy is highly complex, and often presents a mental health professional with an extremely challenging, conventionally difficult to grasp, labyrinth of treatment-plan routes, including, within such a labyrinth, a complicated landscape of potential alternative treatment modalities. This labyrinth is further complicated, in many instances, by a need, during plan implementation, to obtain for evaluation a clear and comprehensive review of the history of plan implementation to date, including one which includes both patient- and professional-perceived, cogent and terse assessments of actual, to-date, plan effects, and perhaps also of a potential need to make treatment-plan route changes based upon such a review.

Creating and seeing comprehensively such a history, especially in the context of considering, at all times, chosen-route viability, and weighing this against alternative-plan options, can be bewildering, extraordinarily time consuming, and filled with unwanted opportunities inadvertently to misjudge appropriate plan activities.

The present invention addresses this therapy realm in a highly intuitive, and greatly clarifying, manner—a manner enabling a mental health professional, in the important context of performing in a working collaboration with a patient, to plan, implement and follow, i.e., track, and, where necessary, to modify, a mental health treatment program for the patient, ultimately, in conditions which are much simpler and more manageable than, and much more improved in relation to, those today conventionally experienced in treatment-plan/program design and execution.

In this setting, the present invention features a software-driven, electronic-information-system-supported methodology, and an associated system, referred to as a mental health therapy-assistance system, for improving the mental health therapy process, or program, for a patient. Access to the relevant, invention-featured, information-system software may, selectively, take place through a visual-user-interface-presented web browser. The methodology, as between a mental health professional and a patient, begins in an initially established, and centrally important, in-depth, patient/mental-health-professional collaboration dialogue, preferably encouraged by the practice personalizing nature of the system software of the present invention, and initiated by the professional, with a focus, inter arta, on capturing in that dialogue information from a patient to determine a patient's mental-health-related symptoms, and leads, from and within this collaborative dialogue, to the establishing of a highly individualized treatment strategy to address such symptoms. This invention, as mentioned above, importantly bypasses the common, conventional/traditional creation, by a mental health professional, of a “top-down”, general diagnosis of one or more symptoms—a practice which often occurs without careful consideration being directed to the patient's objectives, and without ultimately tailoring a treatment strategy to the patient's goals.

Instead, this invention relies on selections made by a mental health professional, in collaboration with a patient, and use of information-system accessible software, to develop narrowly-focused treatment strategies for addressing the patient's mental health condition. In addition, this invention, through key, electronic-information-system involvement, sorts mental health symptoms in a manner that reduces the human-error-related potential for misdiagnosis of one or more mental health-related symptoms. The method and system of the current invention further provides, at user request, and through the provided system software, abbreviated teachings, in the forms of short, field-recognized, mental health aspect summaries, with highlights, and principally, though not exclusively, to a mental health professional, in relation to the building, implementing, modifying, and tracking the history and progress, of mental health treatment plans for mental health patients.

As mentioned above, the field of mental health treatment is highly complex, with many considerations, including taking into account the severity of individual symptoms experienced by a patient, as well as implementing a treatment strategy that appropriately addresses the individual goals of a patient. A common, conventional, however often problematically inaccurate, process of mental health diagnoses involves establishing a treatment strategy to address a particular symptom without including and considering patient-expressed, long-term goals and objectives. By not considering, and even centralizing, the individualized goals and objectives of a patient, a health care provider may easily implement a general, and unfortunately misaligned, diagnosis to a specialized problem. In addition to being frequently under-specialized, conventional treatment strategies often require high amounts of repetitive, face-to-face time, and associated payments, for general mental health professional and patient conversations and evaluations. In a time when health care costs are high, if not prohibitive, for many people, patients increasingly expect health care to be efficiently effective, and as simple as possible, so that expense can be minimized.

The present invention, by expressly accounting for the long term goals and objectives of the patient prior to establishing a therapeutic solution, will greatly reduce excess expenditures due to inaccurate, over-general and under-specific therapy solutions. In addition, the proposed, and centrally important, electronic access to an associated electronic information system, and relevant, invention-proposed, mental health software, facilitates use by both patients and professionals and is able, in many ways, to be a substitute for post-treatment-initiation conversations and evaluations.

As mentioned, this invention accomplishes incorporating long-term goals and objectives of a patient into a highly-specific and efficient, visual-interface-interactive, planning-thought-sorting electronic-information-system in the process of formulating a mental-health treatment strategy. In one embodiment of the method of the invention, the invention method is made available to a mental health professional through a software-based, electronic-information system, and perhaps through an appropriate web interface, implemented in a manner such that the patient and professional may interact with the system and its associated software via an interactive, visual user interface, or computer display. The first interaction between a user and the interface involves answering a series of software-system furnished, planning-thought, hierarchically-sorted, attention-focusing, and appropriately narrowing, questions seeking the inputting of answers related to the patient's mental health symptoms, so as to categorize the patient's problems into at least one reportable umbrella, or overarching, type of mental health issue. Examples of such umbrella issues include anxiety disorders and social disorders. Prior to this first interaction, the mental health professional and the patient, engage in a comprehensive discussion, or dialogue, to identify patient-perceived behavioral and mental health problems.

Following the presentation of the umbrella issues, the professional and patient select at least one umbrella issue which the professional and the patient view as of greatest importance to treat. After this selection is made, the system provides the professional with additional questions, seeking answers, in order to categorize, and further target/narrow, the specific symptoms which are of greatest importance to the patient. After this second round of questions/answers, the professional and patient select, from a comprehensive, previously-compiled, list of mental health symptoms, also known as a symptom cluster, at least one symptom on which to focus treatment. An important advantage of this symptom-selection step is that it avoids an error associated with traditional diagnosis that ignores the severity of various experienced symptoms, and that, in turn, may focus on symptoms which are either less important or less severe for the individual patient, instead of on more prevalent and concerning symptoms.

For each chosen symptom, the system presents, to the user, through a question- and answer interactive dialogue, a variety of long-term goals associated with treating, or overcoming, such a mental health symptom. The long-term goals, designed to provide the user options to pinpoint an individual patient's desired outcome of treatment greatly improves the focus and the effectiveness of a resulting therapeutic strategy.

The system-implemented method, following the selection of each long-term goal for each symptom, provides the user with a list of long-term goal-associated objectives. In other words, the user is then presented with several options, in the form of objectives, to choose from, further to discover, and focus upon, both the desires of the individual patient, and the resulting treatment strategy. For each one long-term-goal selected, the user may select more than one of the objective options to achieve the selected, long term goal. Each objective, generally, will describe (1) who will perform what tasks, (2) what tasks will be performed, and (3) the timeline for completing such tasks.

Finally, following each objective selection, the system-presented method establishes at least one treatment strategy for the patient, tailored to achieve the patient's long-term goal(s) and objective(s) for a given symptom. At this point, one embodiment of the invention presents pre-programmed treatment strategies which are automatically displayed once all the mentioned selections are made.

Another helpful element of the current invention is that in some embodiments, at appropriate times during the creation and implementation of the treatment strategy, the system, at the command/request of a user, generates presentations of highlighted and abbreviated, recognized mental health teachings in the forms of short summaries with appropriate highlights associated with a particular patient symptom. Such generated summaries efficiently help guide the professional, and patient, to understand and further select appropriate target symptoms.

The invention also includes a tracking and associated display interface which allows both the patient and the mental health professional easily to view the patient's treatment history and degree of progress. One such example of a manner to track patient progress is a numerical rating parameter, for both the patient and the professional to rate the level of the patient's achievement relating to one symptom, goal, objective, or therapy strategy.

Such information is further compiled as the treatment continues to track both the patient's and the professional's evaluations of success or progress. This data is further compiled into a visual, graphical format that shows both the patient's and the professional's numerical evaluation, or rating, of success over time, wherein the time is associated with various stages of treatment. An advantage to the tracking ability of this invention is that it places ultimate supervision and control of and over a given treatment strategy in the hands of a mental health professional, while using the electronic-information system to maximize the system and the method of the invention's electronic accuracy and compiling abilities. Such a rating system also allows the patient and the mental health professional, together, to input electronically, and compare their perceptions of treatment progress. The invention also presents a hierarchically-organized presentation of the selected symptoms, long-term goals, objectives, and therapy strategies, in a manner that allows a user to both keep track of his or her tasks, and to indicate, on the interface, his or her progress for each task.

Such interface-presenting visual representations of the therapy strategy, in the current invention, may also be printed into a tangible report. This form of a report may be individually created such that only selected portions (for example, the selected long-term goals and associated evaluations) are shown. The importance of such tangible reports is that they can easily be stored and organized for the benefit of the mental health professional alone with his or her employer, or for other purposes.

From one structural-embodiment point of view, the invention can be described as an interactive, electronic-information-handling system for improving the therapy processes applied by mental health professionals to mental health patients in a manner that increases the focus on an individual patient's desires, and the ability for effective therapy monitoring, including
(a) system-implemented, information-processing software programmed to accept, and progressively to guide in a focusing fashion, as appropriate, information input by a mental health professional about the symptoms and therapy goal desires of an individual mental health patient, and from such information, to create a patient-driven, individualized therapy plan for the patient, and
(b) system-implemented monitoring software operatively connected to the information-processing software, programmed to accept therapy plan implementation and therapy result evaluations from the mental health professional and the patient, reflecting the progress of the therapy plan, and to display such evaluations and therapy results in a visual, graphical format.

From another structural perspective, the invention is characterizable as being a traditional-diagnosis-bypassing, mental health therapy-assistance system for aiding a mental health professional, working in guided collaboration with a mental-health patient, in the designing, building, implementing, modifying, and tracking, of a patient-personalized, behavioral-transformation therapy plan for the patient, including

(1) a patient-and-professional-accessible, interactive, electronic information system,

(2) an interactive user interface operatively connected to the information system and available to the mental health professional and the patient for interaction with the information system, and

(3) electronic structure contained within the information system, programmed with mental-health, behavioral-transformation information-capture, and related, professional and patient interactive-support, software, possessing, as operatively interconnected, software-based, electronic substructures,
(a) first electronic substructure associated with the establishment of a mental-health, behavioral-transformation, therapy-plan collaboration between the patient and the professional,
(b) second electronic substructure associated with, and for communicating interactively regarding, information respecting a professional-and-patient collaboration dialogue engaged in to identify (1) perceived, patient-associated behavioral and mental health problems, (2) potentially perceived-problem-linked, discernable symptoms, and (3) patient-expressed goals associated with the identified problems and symptoms,
(c) third electronic substructure associated with, and for communicating interactively regarding, information respecting dialogue-outcome delivery, from the professional to the patient, of a defined symptom cluster including the identified, potentially problem-linked, discernible symptoms,
(d) fourth electronic substructure associated with, and for communicating interactively regarding, information respecting a collaboratively chosen, preselected maximum number of the cluster-presented symptoms, and for each such chosen symptom, at least one collaboratively established, associated long-term goal aimed at addressing the chosen symptom,
(e) fifth electronic substructure associated with, and for communicating interactively regarding, information respecting collaboratively chosen, goal-associated objectives, and
(f) sixth electronic substructure associated with, and for communicating interactively regarding, information respecting collaboratively established therapeutic strategies.

These and other features and advantages of the invention will become more fully apparent as the detailed description of it which follows below is read in conjunction with the accompanying drawings.

### DESCRIPTIONS OF THE DRAWINGS

FIG. 1 is a simplified, block/schematic diagram illustrating, from two different points of view that are described below, two, preferred, high-level-view manners of practicing the methodology of the present invention.

FIG. 2 is another, simplified, block/schematic diagram illustrating another, somewhat more detailed, preferential manner of practicing the methodology of the invention.

FIG. 3 presents still another, simplified, block/schematic diagram, here picturing an additional methodology practice involving, during the implementation of a mental health therapy plan, the collecting and presenting, in a graphical and numeric display fashion, as on an electronic user-display-interface, periodic “numeric value” appraisals made by both a mental health professional and a patient regarding respectively perceived progress of the plan with respect to some chosen plan aspect.

FIG. 4 presents a representative display of the type mentioned in the description of FIG. 3.

FIG. 5 is a simplified, block/schematic diagram illustrating yet another modified form of the invention methodology, specifically relating to the presentation, during, the implementation of a mental health treatment plan, and for example on an electronic user interface, for use, principally, though not exclusively, by the mental health professional, of selected, recognized, summarized and appropriately highlighted, mental-health-field teachings relating to a then relevant patient symptom.

FIG. 6 offers a block/schematic diagram illustrating yet another modified, architectural form of the invention methodology which accommodates the selective generating of a status and/or history report of one or more facets of an underway, mental health therapy plan.

FIG. 7 illustrates, in simplified, block/schematic form, one high-level picture of the system of the present invention.

FIG. 8 further illustrates, and in a more detailed fashion, the mental health therapy-assistance system of the invention.

### DETAILED DESCRIPTION OF THE INVENTION

As a general-discussion precursor to exploring the invention particulars which are pictured in the illustrative drawings, and as has been mentioned above, the system and methodology of the present invention involve promoting an improved-accuracy approach toward the design and implementation of a mental health therapy program for dealing with issues involving a mental health patient, which approach focuses intently, and in a decidedly traditional-diagnosis-bypassing manner, on patient-expressed therapy goals. It is this important, in a manner of speaking “patient-driven” approach—featured in and by the present invention—that results successfully in the designing, and ultimately the implementing, of an effective mental health therapy plan for a patient.

For example, in the practice of the present invention, and with an appropriate collaborative dialogue encouraged and undertaken by a mental health professional in association with his or her mental health patient, the patient, when asked to express therapy goals might say something like:
“I feel that I lead, because of certain fears, a very lonely and hidden life. I stay in, and at home, most of the time, because I am so uncomfortable being out in the public and in the presence of many other people.
“I need, of course, and usually several times each week, to go to my neighborhood supermarket to buy food and other supplies, and I′m usually so bothered, nervous, and stressed by the prospect of actually doing that, that I really most always go very late at night, or very, very early, just after midnight, in the morning, in other words at times when I hope to avoid having to make any significant contact with other people around me.
“You ask me if there is anything, or if there are any things, which I can think of as we're talking here that could be changed, that is, that I would like to change, and that, if changed, would so improve my spirit and sense of well-being that I could feel so much more comfortable with my life. Yes, there is something, and it would be to have a sense of real, non-fearing comfort just to go several times a week to my supermarket at more normal times, and not fear encountering other people. I like food shopping, and if that change could take place, I would feel that my life and mood have been greatly enhanced. I think I could feel so much more relaxed—safe.”

This, then, is one poignant illustration of a patient expression of a therapy goal—a simple pleading—which, if properly addressed, as by thoughtful practice of the present invention, offers the possibility for making a significant mental health improvement in the subject patient's state of being. No matter what traditional mental health characterization may be applied as an identifier for this situation, it is very clear that a mental health professional, given this patient statement of a goal, has an opportunity to work with that patient in a significant and very focused manner to improve the patient's mental health condition just by addressing a simple and quite singular request.

To do this, of course, a professional must focus upon, and adhere to, a therapy-plan design, followed by an appropriate plan implementation, all integrated in a collaborative manner with the patient's expressed wishes, to carry out what will probably turn out to be an extremely effective and important, and perhaps relatively simply effected, change in that patient's life.

The present invention offers a methodology and an associated system aimed at helping, and in fact focusedly driving, this to happen.

The invention, as has been pointed out, directs attention importantly on a “throughout”, professional/patient collaboration which is aimed significantly toward realizing, for implementation a mental health therapy plan, which, in all of its aspects, is based upon goal-expressed input from a patient. Preferably, therefore, the system software which is employed throughout practice of the invention has been carefully prepared by the designer of that software, knowledgeable about the field of mental health therapy, to have a very evident patient-personalization characteristic, or “personality”, which, from the outset of practice of the invention, and in conjunction with each accessing of the invention software, clearly encourages professional and patient collaborational focus upon a patient's expressions of needs, goals, etc.

In this context, the system and methodology of the invention are designed carefully to project, through a user-display interactive interface, which might take the form of a web browser, focusing guidance for all of the stages and steps involved in the designing and implementing of a mental health therapy plan. This activity is based upon receiving, in the employed electronic-information system, professional/patient (user) dialogue information, and returning, in relation thereto, stages of guiding questions for the receipt of planning, etc. answers, followed by further focusing questions (and received answers) in order to home in on plan personalization.

The present invention does not involve language construction, or order or focus, of specific question texts built into the system software which handles the flow of user/system dialogue-associated information via an interactive user interface. These language, etc., matters are understood by us, and will be understood by those skilled in the art, to be freely and variously creatable by, and/or under the guidance of, skilled mental health practitioners. The same is true with regard to the selections, and preferably shortened contents, of highlighted and abbreviated, summaries of field-recognized teachings respecting mental-health-patient symptoms, etc., that may be displayed selectively to a system user during and throughout practice of the invention.

In the practice contemplated by the invention, important therapy-design, etc. stages—the array mentioned above—understandable as being relatively simple and straightforward, and suitably comprehensive, in carrying out the methodology of the invention, are directed principally to a relatively small handful of key considerations which can be expressed as: patient symptoms and perceivably associated problems; relevant clusters of such symptoms and problems; goals for therapy accomplishment associated with symptoms and perceived problems; chosen and stated objectives linked to identified therapy goals; and finally, relevant implementation strategies which seem to be best suited to meeting stated objectives.

As the detailed description of the invention now proceeds with specific illustrative discussions relating to the several drawing figures, these just-presented precursor thoughts that characterize the system and methodology of the invention will become apparent.

Turning now to FIG. 1, shown generally at 10, is a block-diagram representation of two, preferred, high-level architectural views of the methodology of the present invention. We intend herein that this figure be read in two different ways, one of which effectively outlines the methodology of the invention from the viewpoint of a designer—starting with, and then flowing from, creation and configuration of system software, and the other of which relates to the viewpoint of a user of the methodology of the invention, as, for example, from the viewpoint of a mental health professional working with a collaborating mental health patient. With respect to the first-mentioned “reading” of FIG. 1, and of the three blocks, 12, 14, 16, pictured in this figure, block 12 should be read in relation to the non-parenthetical term “Configuring” presented in this block. The other reading should focus on the parenthetical word “Accessing” presented in block 12.

Considering the first-intended reading of FIG. 1, this figure illustrates an electronic-information-system-supported, traditional-diagnosis-bypassing, software-based method 10 for improving the accuracy of a mental health therapy program for a mental health patient through incorporating, centrally, an individual mental health patient's expressed therapy goals.

Block 12 represents the first, general step of this method which includes both configuring, and making available to a mental health professional and a mental health patient, through a user-interface-accessible, interactive electronic information system, appropriate method-implementing, mental health information-capturing and guiding software characterized with system-software-evident, patient-personalizing, mental-health-therapy-associated content.

Following, and in association with, such configuring and making available, and in relation to the mentioned, system-software-evident patient-personalizing-content, the next step in the method, shown by Block 14, involves encouraging (a) the establishment of a collaborative dialogue between the patient and the professional to capture information relating to the patient's mental health symptoms and associated, patient-expressed therapy goals. Included within the practice represented by Block 14, and following the capturing of information, the professional enters the related dialogue information into the system.

Block 16 represents a final step in the currently being described method of the invention, wherein, progressing from the system-entered, collaboration-dialogue information (Block 14), and in a system-interactive, appropriately recurrent (a) question, (b) response-entry, (c) further question, and (d) further response-entry manner, driven by the system software, which manner employs entered information and entered-information-triggered, software-based questions in a traditional-diagnosis-bypassing mode that centrally recognizes the patient's expressed therapy goals, at least one patient-specific, mental health therapy is collaboratively designed, built, and implemented, based upon the patient's stated therapy goals.

According to the second-mentioned reading of FIG. 1, here shown at 10 is an electronic-information-system-supported, conventional-diagnosis-bypassing, software-based method for improving the accuracy of a mental health therapy program for a mental health patient through incorporating, centrally in all phases linked with such a program, an individual mental health patient's expressed therapy goals including: (a) accessing (Block 12) an interactive electronic information system which is software-programmed with appropriate, information-capturing and related-question-based, focusing and guiding software associated with patient-personalizing mental health therapy planning, structuring and implementing;

(b) encouraging (Block 14) the establishment of a collaboration dialogue between patient and professional to capture information relating to the patient's mental health symptoms and associated, patient-expressed therapy goals, and collaboratively entering related dialogue information into the system; and

(c) based upon such information entering, and through professional-and-patient interactive engagement with the system, and functioning expressly in a conventional-diagnosis-bypassing, question and answer mode which is bottomed centrally upon recognizing the patient's expressed therapy goals, collaboratively designing, building, and thereafter implementing (Block 16), a patient-specific therapy.

Looking now at FIG. 2, this figure furnishes another, more detailed, architectural view of a manner for practicing the methodology of the invention, shown generally at 18 in a composite block diagram including eight blocks connected together with single-headed arrows designating an order of the relevant methodological steps. These steps, as a whole, represent an electronic-information-system-supported method for aiding a mental health professional, working in guided collaboration with a mental health patient, in the creation, ultimately, of a highly patient-personalized, behavioral-transformation therapy plan for addressing the patient's mental or behavioral health problems.

Block 20 in methodology 18 represents the first, foundational step of this methodology, a step which involves establishing a mental-health, behavioral-transformation, therapy plan collaboration between the patient and the professional.

What next follows, represented by Block 22, is an engagement, by the professional and the patient, in a collaboration dialogue, and a related employment of an interactive user interface, associated with the system of the invention, which allows for interactive communication from the patient and professional to the system, and from the system to the patient and professional, regarding a flow of input and output information associated with the collaboration dialogue between these two persons. The purpose of the collaboration dialogue is to identify: 1) perceived, patient-associated behavioral and mental health problems, 2) potentially perceived-problem-linked, discernible symptoms, and 3) patient-expressed goals associated with the identified problems and symptoms.

The step represented by Block 22 is important in the practice of the invention because within it lies the foundation upon which all information used to develop a mental health therapy plan rests. The associated dialogue, significantly, provides the professional with personalized information, directly from the patient, about what he or she, i.e., the patient, identifies to be, for instance, the most severe of experienced mental or behavioral health symptoms, perhaps, though not necessarily, a singular symptom, and the most important patient-imagined outcome of professional treatment. The dialogue also develops information respecting patient- and professional-perceived problems that are, apparently, linked with the identified symptoms.

Following such a dialogue, Block 24 represents the next step in methodology 18, wherein, based upon the dialogue which has taken place between the professional and the patient, the professional, with system-software assistance, delivers, or communicates, to the patient, what is referred to herein as a defined symptom cluster. Such a symptom cluster includes all identified, potentially problem-linked, discernible symptoms. Question and answer assistance and support provided by the system software regarding this topic, tied to collaboration-dialogue input information, aid in formulating a relevant symptom cluster for further consideration in the designing of a patient-personalized therapy plan

After delivery of a symptom cluster (as represented by Block 24), the step of Block 26 comes in to play. This step involves a collaborative (professional and patient) selection, or a choosing, for attention, of at least one of the cluster-presented symptoms. Similar to the engaging of a collaboration dialogue between the patient and the professional, this choosing step greatly focuses and specializes the later-established treatment therapy to address symptoms selected collaboratively based on the personal desires of the patient, and the associated understanding of the professional.

Following the Block-26 step involving the choosing of symptoms, or the choosing of at least of one symptom, for focused attention, methodology activity represented by Block 28 comes into play. According to Block 28, what takes place is the establishing of at least one, symptom-associated, long-term goal for each chosen symptom. In this step, the patient and professional are able to choose, collaboratively, the best long-term goal options that most appropriately represent the desires of the patient.

After the establishment of at least one long-term goal in the methodology step associated with Block 28, Block 30, which follows next, represents the choosing of at least one, long-term, goal-associated objective for each established long-term goal. Such goal-associated objectives, even if only one in number, further increase(s) the focus of the later-established therapy strategy to center on the desires of the patient.

Block 32 represents the next methodologic step, and namely, a step involving the establishing of at least one therapeutic strategy associated with each chosen objective. Block 34 completes an expression of methodology 18. It refers simply to the undertaking of appropriate implementation of the Block-32 established strategy or strategies, designed now to effect patient treatment.

Throughout practice of the methodology illustrated in FIG. 2, as well as throughout practices of the two methodologies described in relation to FIG. 1, patient/professional collaboration reigns, and this important and central patient-focused collaboration condition featured by the present invention assures focused, effective, sensitive, patient-personalized mental-health-plan designing, building and implementing.

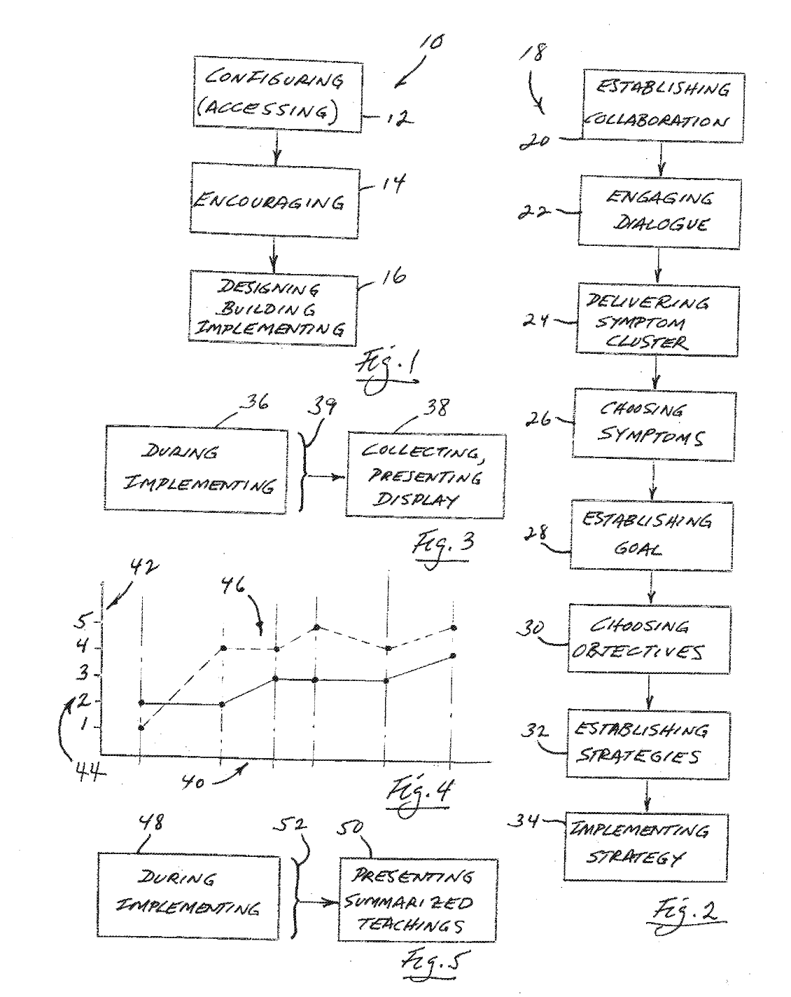

Turning attention now to FIGS. 3-5, inclusive, the methodology of the invention may include further steps, as illustrated in these four figures. FIG. 3, containing two blocks, Blocks 36 and 38, shows that at any time during implementation of the method of the invention, a graphical/numerical display of collected professional/patient perceptions of selected-topic therapy progress may, selectively, be displayed. A bracket 39 is included in this figure to illustrate that the presentations, or summarized teachings, can be presented, as just stated, at any time during method implementation.

FIG. 4 shows an illustrative example of such a display. This example represents, on the x-axis which is generally designated 40, days, or instances in time, when the patient and/or the professional evaluates the progress of therapy. The Y-axis, which is generally designated 42, represents an example of a numerical rating system 44, wherein the patient and/or the professional selects a whole number in the illustrated range of 1 to 5, with 1 representing a low, or otherwise dis-satisfactory, perception of therapy progress at that given date. The reported evaluative ratings can then be compiled in a manner that shows both the patient's, and the professional's evaluations over time. FIG. 4 shows, generally at 46, two lines that separately represent the numerical evaluations of the patient and the professional throughout the period of therapy represented in this figure. Herein, the dashed line represents the patient's evaluations, and the full line represents the professional's evaluations.

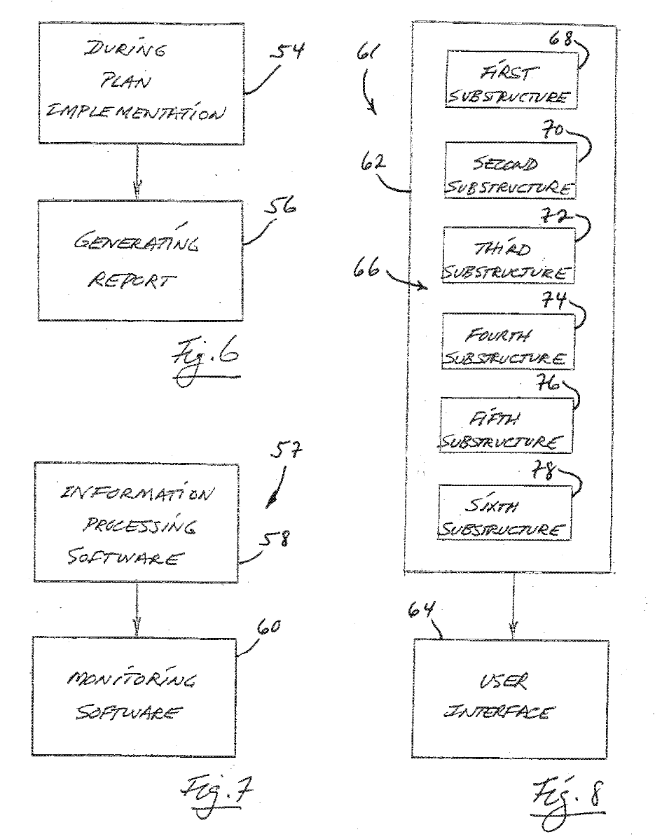

Turning now to FIG. 5, this two-part block diagram represents an additional methodological step wherein, during any appropriate stage of therapy-plan implementation, Block 48, the “during-implementation”, system-supported method of the invention, provides access to system-based presentations of highlighted and abbreviated, recognized mental health teachings, Block 50. Bracket 52 is included in this figure to illustrate that the presentations, or summarized teachings, can be presented at any time during method implementation. Such presentations are short summaries with appropriate highlights associated with a patient symptom, for teaching and helping the mental health professional in the implementation of a plan.

FIG. 6 is two-block diagram which represents a further aspect of the method of the present invention, wherein the first block, Block 54, represents the overall implementation of the method of the invention, and the second block, Block 56, shows a further step wherein a plan-implementation, or a portion of a plan implementation, report is created. Such a report is generated based on any of the selected input or output information, and may typically be related to at least one of the patient-related symptoms, long-term goals, objectives, or strategies that are part of an “underway” plan.

Turning attention now to FIG. 7, this figure pictures generally at 57, in two, operatively connected Blocks, 58, 60, one embodiment form of the system of the invention, referred to as an interactive, electronic-information-handling system. As illustrated and described in previous sections of this disclosure, the system of the invention, as represented in embodiment form 57, serves to improve the therapy processes applied by mental health professionals to mental health patients in a manner that increases the focus on an individual patient's desires, alongside providing the ability for effective treatment monitoring. In this illustrated embodiment, Block 58 represents information-processing software, and Block 60 represents appropriate, system-implemented monitoring software.

Block 58 is structured to accept, and progressively to guide in a focusing, or narrowing, fashion, information input by a mental health professional about the symptoms and therapy-goal desires of an individual mental health patient. From such information, the Block 58 further creates a patient-driven, highly-individualized therapy plan for the patient.

Block 60, which is operatively connected to Block 58, as mentioned, is programmed to accept therapy plan implementation selections, made by the professional and the patient, and therapy result evaluations that reflect the progress of the therapy plan. This block is also programmed to display such evaluations and therapy results in a visual/graphical format on an appropriate user interface display (not shown in FIG. 7).

FIG. 8 illustrates, at 61, another structural embodiment form of the present invention, referred to as a traditional-diagnosis-bypassing, mental health therapy-assistance system for aiding a mental health professional, working in guided collaboration with a mental-health patient, in the designing, building, implementing, modifying, and tracking, of a patient-personalized, behavioral-transformation therapy plan for the patient.

Assistance system 61 includes:

a patient-and-professional-accessible, interactive, electronic information system (Block 62),

an interactive user interface (Block 64) which is operatively connected to the information system, and available to the mental health professional and the patient for interaction with the information system, and

a six-block-represented electronic structure (generally indicated at 66) contained within information system 62, appropriately programmed with mental-health, behavioral-transformation information-capture, and related, professional and patient interactive-support, software, including, as suitably operatively interconnected, software-based, electronic substructures,

(a) first electronic substructure (Block 68) associated with the establishment of a mental-health, behavioral-transformation, therapy-plan collaboration between the patient and the professional,

(b) second electronic substructure (Block 70) associated with, and for communicating interactively regarding, information respecting a professional-and-patient collaboration dialogue engaged in to identify (1) perceived, patient-associated behavioral and mental health problems, (2) potentially perceived-problem-linked, discernable symptoms, and (3) patient-expressed goals associated with the identified problems and symptoms,

(c) third electronic substructure (Block 72) associated with, and for communicating interactively regarding, information respecting dialogue-outcome delivery, from the professional to the patient, of a defined symptom cluster including the identified, potentially problem-linked, discernible symptoms,

(d) fourth electronic substructure (Block 74) associated with, and for communicating interactively regarding, information respecting a collaboratively chosen, preselected maximum number of the cluster-presented symptoms, and for each such chosen symptom, at least one collaboratively established, associated long-term goal aimed at addressing the chosen symptom,

(e) fifth electronic substructure (Block 76) associated with, and for communicating interactively regarding, information respecting collaboratively chosen, goal-associated objectives, and

(f) sixth electronic substructure (Block 78) associated with, and for communicating interactively regarding, information respecting collaboratively established therapeutic strategies.

The method and the system of the invention, therefore, as fully described herein, provides distinct and highly-effective advantages, to both a mental-health patient and mental-health professional. Both the method and the system of the invention avoid the traditional, often inaccurate, manner of diagnosis, and achieve much greater effectiveness with a highly-patient-focused way to develop mental health treatment plans. In particular, the method of the invention importantly encourages the establishment of a patient-professional dialogue from which the mental health therapy is derived. The system of the invention also, by providing an interactive user interface that facilitates communication between the user and the system, is specifically designed to accept dialogue-based information to create a behavioral-transformation therapy plan for an individual patient.

While preferred embodiments of, and manners of practicing, the invention, and certain modifications thereof, have been set forth herein, we appreciate that other variations and modifications may come to the minds of those skilled in the art, and that these variations and modifications may be made without departing from the spirit of the invention.

We claim:

1. An electronic-information-system-supported, traditional-diagnosis-bypassing, software-based method for improving the accuracy of a mental health therapy program for a mental health patient through incorporating, centrally, an individual mental health patient's expressed therapy goals comprising
configuring, and making available to a mental health professional and a mental health patient, through a user-interface-accessible, interactive electronic information system, appropriate method-implementing, mental health information-capturing and guiding software characterized with system-software-evident, patient-personalizing, mental-health-therapy-associated content,
in association with said configuring and making available, and in relation to the mentioned, system-software-evident patient-personalizing-content, encouraging (a) the establishment of a collaboration dialogue between patient and professional to capture information relating to the patient's mental health symptoms and associated, patient-expressed therapy goals, and (b) the entry of related, collaboration-dialogue information into the system, and
progressing thereafter from this system-entered, collaboration-dialogue information, and in a system interactive, question and response-entry manner which uses the information in a traditional-diagnosis-bypassing mode that centrally recognizes the patient's expressed therapy goals, collaboratively designing, building, and thereafter implementing, a personalized, patient-specific, mental health therapy.

2. The method of claim 1, wherein said making available through a user-interface takes place through a web browser.

3. An electronic-information-system-supported, conventional-diagnosis-bypassing, software-based method for improving the accuracy of a mental health therapy program for a mental health patient through incorporating, centrally in all phases linked with such a program, an individual mental health patient's expressed therapy goals comprising
accessing an interactive electronic information system which is software-programmed with appropriate, information-capturing and related-question-based, focusing and guiding software associated with patient-personalizing mental health therapy planning, structuring and implementing,
encouraging the establishment of a collaboration dialogue between patient and professional to capture information relating to the patient's mental health symptoms and associated, patient-expressed therapy goals, and collaboratively entering related dialogue information into the system, and
based upon said entering, and through professional-and-patient interactive engagement with the system, and functioning expressly in a conventional-diagnosis-bypassing, question and answer mode which is bottomed centrally upon recognizing the patient's expressed therapy goals, collaboratively designing, building, and thereafter implementing, a patient-specific therapy.

4. The method of claim 3, wherein said accessing in performed through a user-interface-presented web browser.

5. An electronic-information-system-supported method for aiding a mental health professional, working in guided collaboration with a mental-health patient, in the designing, building, implementing, modifying, and tracking, of a patient-personalized, behavioral-transformation therapy plan for the patient, said method, under collaboration guidance furnished by the mental-health professional, comprising
in the context of a patient-and-professional-accessible, electronic information system offering an interactive user interface with respect to which appropriate system input and output information flow takes place regarding all of the below-stated methodology steps, and which system is at least one of (a) in possession of, and (b) linked with, appropriate mental-health behavioral-transformation programming, along with associated mental-health support information, and which wherein the system and support information are dependently and interactively used and implemented in the practice of the method, establishing a mental-health, behavioral-transformation, therapy-plan collaboration between the patient and the professional,
following said establishing, engaging in a professional-and-patient collaboration dialogue to identify (a) perceived, patient-associated behavioral and mental health problems, (b) potentially perceived-problem-linked, discernable symptoms, and (c) patient-expressed goals associated with the identified problems and symptoms, based upon the outcome of the engaged-in dialogue, and guided by the therein patient-expressed goals, effectively delivering, from the professional to the patient, a defined symptom cluster including the identified, potentially problem-linked, discernible symptoms,
collaboratively choosing to work on up to a preselected maximum number of the cluster-presented symptoms,
for each chosen symptom, collaboratively establishing at least one, associated long-term goal aimed at addressing the chosen symptom,
for each symptom-associated and collaboratively established long-term goal, collaboratively choosing up to another, preselected number of goal-associated objectives,
for each chosen, goal-associated objective, collaboratively establishing yet another, preselected number of therapeutic strategies, and
selectively, and collaboratively, implementing at least one of the established strategies.

6. The methodology of claim 5 which further includes, during said implementing, collecting, and selectively presenting a graphical/numerical display of, professional/patient perceptions of selected-topic therapy progress.

7. The methodology of claim 5 which further includes, at an appropriate stage of therapy-plan implementation, system-based presenting of highlighted and abbreviated, recognized mental health teachings in the forms of short summaries with appropriate highlights associated with a patient symptom for teaching and aiding the practicing mental health professional in the implementation of the plan.

8. The methodology of claim 5 which further includes, selective therapy-plan report-generating of the previously-input and output information, related to at least one of patient-related symptoms, long-term goals, objectives, and strategies.

9. An interactive, electronic-information-handling system for improving the therapy processes applied by mental health professionals to mental health patients in a manner that increases the focus on an individual patient's desires, and the ability for effective therapy monitoring, comprising
system-implemented, information-processing software programmed to accept, and progressively to guide in a focusing fashion, as appropriate, information input by a mental health professional about the symptoms and therapy goal desires of an individual mental health patient, and from such information, to create a patient-driven, individualized therapy plan for the patient, and
system-implemented monitoring software operatively connected to said information-processing software, programmed to accept therapy plan implementation and therapy result evaluations from the mental health professional and the patient, reflecting the progress of the therapy plan, and to display such evaluations and therapy results in a visual, graphical format.

10. A traditional-diagnosis-bypassing, mental health therapy-assistance system for aiding a mental health professional, working in guided collaboration with a mental-health patient, in the designing, building, implementing, modifying, and tracking, of a patient-personalized, behavioral-transformation therapy plan for the patient, comprising
a patient-and-professional-accessible, interactive, electronic information system,
an interactive user interface operatively connected to said information system and available to the mental health professional and the patient for interaction with the information system, and
electronic structure contained within said information system, programmed with mental-health, behavioral-transformation information-capture, and related, professional and patient interactive-support, software, including, as operatively interconnected, software-based, electronic substructures,
(a) first electronic substructure associated with the establishment of a mental-health, behavioral-transformation, therapy-plan collaboration between the patient and the professional,
(b) second electronic substructure associated with, and for communicating interactively regarding, information respecting a professional-and-patient collaboration dialogue engaged in to identify (1) perceived, patient-associated behavioral and mental health problems, (2) potentially perceived-problem-linked, discernable symptoms, and (3) patient-expressed goals associated with the identified problems and symptoms,
(c) third electronic substructure associated with, and for communicating interactively regarding, information respecting dialogue-outcome delivery, from the professional to the patient, of a defined symptom cluster including the identified, potentially problem-linked, discernible symptoms,
(d) fourth electronic substructure associated with, and for communicating interactively regarding, information respecting a collaboratively chosen, preselected maximum number of the cluster-presented symptoms, and for each such chosen symptom, at least one collaboratively established, associated long-term goal aimed at addressing the chosen symptom,
(e) fifth electronic substructure associated with, and for communicating interactively regarding, information respecting collaboratively chosen, goal-associated objectives, and
(f) sixth electronic substructure associated with, and for communicating interactively regarding, information respecting collaboratively established therapeutic strategies.
